(19) United States
(12) Patent Application Publication
(10) Pub. No.: US 2025/0058277 A1
(43) Pub. Date: Feb. 20, 2025

(54) ADVANCED SCALABLE EXTRACELLULAR VESICLE (EV) ISOLATION, SEPARATION, AND CONCENTRATION

(71) Applicant: CORNING INCORPORATED, CORNING, NY (US)

(72) Inventor: Amy Claire Kauffman, Kittery, ME (US)

(21) Appl. No.: 18/710,938

(22) PCT Filed: Nov. 15, 2022
(86) PCT No.: PCT/US2022/049924

§ 371 Date: May 16, 2024

Related U.S. Application Data
(60) Provisional application No. 63/283,012, filed on Nov. 24, 2021.

(51) Int. Cl. B01D 61/18; B01D 61/14; B01D 69/04; C12M 1/00

(52) U.S. Cl. CPC B01D 61/18; B01D 61/145; B01D 69/04; C12M 47/10; B01D 2313/18; B01D 2313/243; B01D 2313/501; B01D 2313/903; B01D 2315/10; B01D 2315/16; B01D 2325/0212

(57) ABSTRACT

A system for separating, isolating, and concentrating extracellular vesicles (EVs) is provided. The system comprises an ultrafiltration device; an isoporous membrane configured for use in the ultrafiltration device; and a collection container for collecting filtrate from the ultrafiltration device. The ultrafiltration device may be configured to perform diafiltration. The ultrafiltration device may comprise a fixed-volume ultrafiltration device. The ultrafiltration device may comprise a tangential flow filtration device. The system may be scalable.

This application claims the benefit of priority under 35 U.S.C. § 119 of U.S. Provisional Application Ser. No. 63/283,012 filed on Nov. 24, 2021, the content of which is relied upon and incorporated herein by reference in its entirety.

### TECHNICAL FIELD

The present disclosure generally relates to extracellular vesicles (EVs) and methods for isolation, separation, and concentration of EVs.

### BACKGROUND

Extracellular vesicles or EVs refer to a population of particles naturally released from cells. EVs are involved with intercellular communication and are involved in many processes in health and disease states such as stress compensation, physiological responses, homeostasis, and various other biological regulatory activities. Because of their therapeutic potential in providing the necessary factors to mediate physiological events, as well as their ability to serve as less invasive diagnostic markers for prognosis of pathological conditions, EVs continue to be of interest to the scientific and medical communities. However, extracellular vesicle research suffers from inconsistent isolation and/or separation methodologies, nomenclature, and lack of standardized data collection and analysis strategies. These challenges limit the ability for the field of EV study and research to mature and progress into a therapeutic possibility.

To date, the most commonly used strategy for processing EVs is differential ultracentrifugation. This technique may be the most commonly used technique due to its easy accessibility in most laboratories. However, differential ultracentrifugation is time-consuming, labor-intensive, and protocols significantly suffer due to the high physical force exerted on the EVs that can affect yield, purity, and physical integrity. To address the shortcomings of differential ultracentrifugation, alternative methods have been developed that utilize capture reagents or physical geometric constraints to drive the isolation. Unfortunately, the proposed alternative methods suffer from drawbacks, such as the significant drawback of not being scalable. As such, a need exists for scalable methods of processing EVs.

### SUMMARY

In an aspect, a system for separating, isolating, and concentrating extracellular vesicles (EVs) is provided. The system comprises an ultrafiltration device; an isoporous membrane configured for use in the ultrafiltration device; and a collection container for collecting filtrate from the ultrafiltration device. In an embodiment, filtrate comprises a concentration of EVs separated and isolated from a biofluid processed by the system.

In an embodiment, the membrane comprises a uniform pore distribution, wherein pores have a uniform pore size. In an embodiment, the uniform pore size is in a range of 30 nm and comprises a pore size distribution where a maximum diameter divided by a minimum diameter is less than 3 nm. In an embodiment, the pore size of the isoporous membrane is selected based on a size of EV particles to be filtered from a biofluid.

In an embodiment, the size of particles to be filtered from the biofluid is in a range of 50 nm to 250 nm. In an embodiment, the size of particles to be filtered from the biofluid is in a range of 80 nm to 150 nm.

In an embodiment, the membrane does not require chemical or surface modifications.

In an embodiment, the membrane is formed of one or more polymer materials. In an embodiment, the one or more polymer materials comprise one or more of poly((4-vinyl)pyridine), poly((2-vinyl) pyridine), poly(ethylene oxide), poly(methacrylates) such as poly(methacrylate), poly(methyl methacrylate), and poly(dimethylethyl amino ethyl methacrylate), poly(acrylic acid), and poly(hydroxystyrene) poly((4-vinyl)pyridine, poly(styrene) and poly(alpha-methyl styrene), polyethylene, polypropylene, polyvinyl chloride, and polytetrafluoroethylene, poly(isoprene), poly(butadiene), poly(butylene), and poly(isobutylene), b-, poly(styrene)-b-poly((4-vinyl)pyridine), poly(styrene)-b-poly((2-vinyl) pyridine), poly(styrene)-b-poly(ethylene oxide), poly(styrene)-b-poly(methyl methacrylate), poly(styrene)-b-poly(acrylic acid), poly(styrene)-b-poly(dimethylethyl amino ethyl methacrylate), poly(styrene)-b-poly(hydroxystyrene), poly(α-methyl styrene)-b-poly((4-vinyl)pyridine), poly(α-methyl styrene)-b-poly((2-vinyl) pyridine), poly(α-methyl styrene)-b-poly(ethylene oxide), poly(α-methyl styrene)-b-poly(methyl methacrylate), poly(α-methyl styrene)-b-poly(acrylic acid), poly(α-methyl styrene)-b-poly(dimethylethyl amino ethyl methacrylate), poly(α-methyl styrene)-b-poly(hydroxystyrene), poly(isoprene)-b-poly((4-vinyl)pyridine), poly(isoprene)-b-poly((2-vinyl) pyridine), poly(isoprene)-b-poly(ethylene oxide), poly(isoprene)-b-poly(methyl methacrylate), poly(isoprene)-b-poly(acrylic acid), poly(isoprene)-b-poly(dimethylethyl amino ethyl methacrylate), poly(isoprene)-b-poly(hydroxystyrene), poly(butadiene)-b-poly((4-vinyl)pyridine), poly(butadiene)-b-poly((2-vinyl) pyridine), poly(butadiene)-b-poly(ethylene oxide), poly(butadiene)-b-poly(methyl methacrylate), poly(butadiene)-b-poly(acrylic acid), poly(butadiene)-b-poly(dimethylethyl amino ethyl methacrylate), and poly(butadiene)-b-poly(hydroxystyrene), poly(isoprene-b-styrene-b-4-vinylpyridine), poly(isoprene)-b-poly(styrene)-b-poly((4-vinyl)pyridine), poly(isoprene)-b-poly(styrene)-b-poly((2-vinyl) pyridine), poly(isoprene)-b-poly(styrene)-b-poly(ethylene oxide), poly(isoprene)-b-poly(styrene)-b-poly(methyl methacrylate), poly(isoprene)-b-poly(styrene)-b-poly(acrylic acid), poly(isoprene)-b-poly(styrene)-b-poly(dimethylethyl amino ethyl methacrylate), poly(isoprene)-b-poly(styrene)-b-poly(hydroxystyrene), poly(isoprene)-b-poly(α-methyl styrene)-b-poly((4-vinyl)pyridine), poly(isoprene)-b-poly(α-methyl styrene)-b-poly((2-vinyl) pyridine), poly(isoprene)-b-poly(α-methyl styrene)-b-poly(ethylene oxide), poly(isoprene)-b-poly(α-methyl styrene)-b-poly(methyl methacrylate), poly(isoprene)-b-poly(α-methyl styrene)-b-poly(acrylic acid), poly(isoprene)-b-poly(α-methyl styrene)-b-poly(dimethylethyl amino ethyl methacrylate), poly(butadiene)-b-poly(styrene)-b-poly((4-vinyl)pyridine), poly(butadiene)-b-poly(styrene)-b-poly((2-vinyl) pyridine), poly(butadiene)-b-poly(styrene)-b-poly(ethylene oxide), poly(butadiene)-b-poly(styrene)-b-poly(methyl methacrylate), poly(butadiene)-b-poly(styrene)-b-poly(acrylic acid), poly(butadiene)-b-poly(styrene)-b-poly(dimethylethyl amino ethyl methacrylate), poly(butadiene)-b-poly(styrene)-b-poly(hydroxystyrene), poly(butadiene)-b-poly(α-methyl styrene)-b-poly((4-vinyl)pyridine), poly(butadiene)-b-poly(α-methyl styrene)-b-poly((2-vinyl) pyridine), poly(butadiene)-b-poly(α-methyl styrene)-b-poly(ethylene oxide), poly(butadiene)-b-poly(α-methyl styrene)-b-poly(methyl methacrylate), poly(butadiene)-b-poly(α-methyl styrene)-b-poly(acrylic acid), poly(butadiene)-b-poly(α-methyl styrene)-b-poly(dimethylethyl amino ethyl methacrylate), and poly(butadiene)-b-poly(styrene)-b-poly(hydroxystyrene).

In an embodiment, the ultrafiltration device comprises an automated ultrafiltration device. In an embodiment, the ultrafiltration device system is configured to control pressure in the system. In an embodiment, the ultrafiltration device is configured to control flow rate of fluid through the isoporous membrane by means of air pressure.

In an embodiment, the ultrafiltration device is configured to perform diafiltration.

In an embodiment, the ultrafiltration device comprises a fixed volume ultrafiltration device. In an embodiment, the fixed volume ultrafiltration device comprises a volume in a range of 50 mL to 200 L.

In an embodiment, air pressure is the separation force. In an embodiment, the system further comprises an air source, air filter, air regulator, or combination thereof.

In an embodiment, the ultrafiltration device comprises a tangential flow filtration device.

In an embodiment, the system further comprises a reservoir for biofluid to be processed. In an embodiment, the system further comprises a pump, flow valve, flowmeter, pressure gauge, or combination thereof.

In an embodiment, the system is scalable.

Additional aspects of the present disclosure will be set forth, in part, in the following detailed description, figures, and claims, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIG. 1 shows a comparison of commercially available membranes and a membrane according to an embodiment of the present disclosure.

FIG. 2 shows an ultrafiltration system according to an embodiment of the present disclosure.

FIG. 3 shows an ultrafiltration system according to an embodiment of the present disclosure.

FIG. 4 shows a graphical image depicting linear regression analysis of specific flux of water over time through a conventional 100 kDa membrane.

FIG. 5 shows a graphical image depicting linear regression analysis of specific flux of water over time through an isoporous 30 nm membrane.

FIG. 6 shows a graphical image depicting linear regression analysis of specific flux of water over time through a conventional 50 kDa membrane.

FIG. 7 shows a graphical image depicting linear regression analysis of specific flux of water over time through an isoporous 10 nm membrane.

FIG. 8 shows a graphical image depicting linear regression analysis of specific flux of water over time through a conventional 30 kDa membrane.

FIG. 9 shows a graphical image depicting linear regression analysis of specific flux of water over time through an isoporous 5 nm membrane.

FIG. 10 shows a graphical image depicting quantitation of collected EVs in the concentrate and permeate according to an embodiment of the present disclosure.

FIG. 11 shows a graphical image depicting quantitation of protein via BCA assay according to an embodiment of the present disclosure.

FIG. 12 shows a graphical image depicting NTA analysis of Vero EV concentration and size from static and perfusion cell culture vessels according to an embodiment of the present disclosure.

FIG. 13 shows a graphical image depicting NTA analysis of Vero EV concentration and size from a perfusion cell culture vessel according to an embodiment of the present disclosure.

FIG. 14 shows a graphical image depicting protein analysis via BCA assay of final EV concentrate and subsequent permeates from static and perfusion cell culture vessels through diafiltration according to an embodiment of the present disclosure.

FIG. 15 shows a graphical image depicting protein analysis via BCA assay of final EV concentrate and subsequent permeates from a perfusion vessel according to an embodiment of the present disclosure.

FIG. 16 shows a graphical image depicting a comparison of protein reduction in final concentrate of Vero produced EVs according to an embodiment of the present disclosure.

FIG. 17 shows a graphical image depicting protein analysis via BCA assay of final EV concentrate and subsequent permeates according to an embodiment of the present disclosure.

FIG. 18 shows a graphical image depicting NTA analysis of Vero EV concentration and size from vessels using fixed volume ultrafiltration devices according to an embodiment of the present disclosure.

FIG. 19 shows a graphical image depicting analysis of concentrated and purified EVs from NTA according to an embodiment of the present disclosure.

FIG. 20 shows a graphical image depicting protein concentration analysis via BCA assay according to the present disclosure.

FIG. 21 shows a graphical image depicting total EV production from a perfusion culture device according to an embodiment of the present disclosure.

FIG. 22 shows a graphical image depicting total EV production from a perfusion culture device according to an embodiment of the present disclosure.

### DETAILED DESCRIPTION

Aspects of the present disclosure are directed to systems and methods to isolate, separate, and/or concentrate extracellular vesicles from biological fluids or biofluids. Conventional techniques typically used in present day settings to capture and purify extracellular vesicles include ultracentrifugation, ultrafiltration, polymer precipitation, immunoaffinity capture, microfluidics, and size-exclusion chromatography. However, disadvantages associated with these techniques include inability to scale up, reliance on a centrifuge, requiring reagents for binding and/or capture, or requiring specialized devices like microfluidic devices or chromatography columns.

In contrast to conventional techniques, the present disclosure provides systems and methods that allow for scalable EV processing from bench-scale investigation to bioprocessing and/or large-scale biomanufacturing. Systems and methods described in the present disclosure comprise an isoporous membrane and an ultrafiltration device. The isoporous membrane is configured to be compatible with, and used in combination with, one or more ultrafiltration devices. In an embodiment, the ultrafiltration device comprises a diafiltration capable device. In an embodiment, the ultrafiltration device comprises a tangential flow filtration device.

Ultrafiltration is a method of filtration that does not require intense time, energy, and equipment to perform. However, the recovery of EVs is dependent on the device and filter selection, which includes material composition, pore size, and surface chemistry. As a result, poor membrane selection may lower EV yield and purity due to the interaction between the EVs and the membranes. For example, the interaction between the EVs and the membranes may generate aggregation of EVs that results in the membrane having blocked pores.

Ultrafiltration is also dependent upon the equipment set-up, driving pressure, and precision of the controller. High pressure may lead to a significant loss in vesicle integrity, damage could be induced by low precision of pressure control, and increased vesicle loss observed due to excessive tubing and connections. Furthermore, ultrafiltration systems used in conventional techniques typically have a fixed working volume. Many of the conventional technique systems used for EVs exhibit a maximum volume of 25 mL, which is a very low working volume. Such a low working volume does not lend itself to easy scale up from bench-top conditions to large scale manufacturing and bioprocessing, wherein liters of biofluid must be processed.

In an aspect, a system for isolating, separating, and/or concentrating extracellular vesicles (EVs) is provided. Due to the nature of the components used in the system of the present disclosure, the system is scalable from bench-top conditions to large-scale bioprocessing or industrial conditions.

In an aspect, a scalable extracellular vesicle processing system is provided that can separate, isolate, and concentrate EVs from biofluids. The system as described in embodiments herein comprises an ultrafiltration device and an isoporous membrane configured for use in the ultrafiltration device. The ultrafiltration device may comprise simple ultrafiltration devices such as a fixed volume ultrafiltration device, a large-scale ultrafiltration device such as a tangential flow filtration apparatus, or any suitable apparatus specific for handling EVs and that is compatible with an isoporous membrane. The system as described in embodiments herein may further comprise a filtrate collection container for collecting EV filtrate. Systems as described herein may be configured for separation of biological nanostructures from biological fluids.

The system for isolating, separating, and/or concentrating EVs comprises an isoporous membrane. In contrast to traditional random porous membranes, isoporous membranes have a tunable polymer chemistry that allows for a membrane with precision pore size wherein the membranes have well-defined micro and nanoscale pore architecture having uniform pore sizes and straight pore channels. The isoporous membrane does not require chemical or surface modifications, is a membrane that is truly isoporous, and is easy-to-use with a variety of ultrafiltration devices.

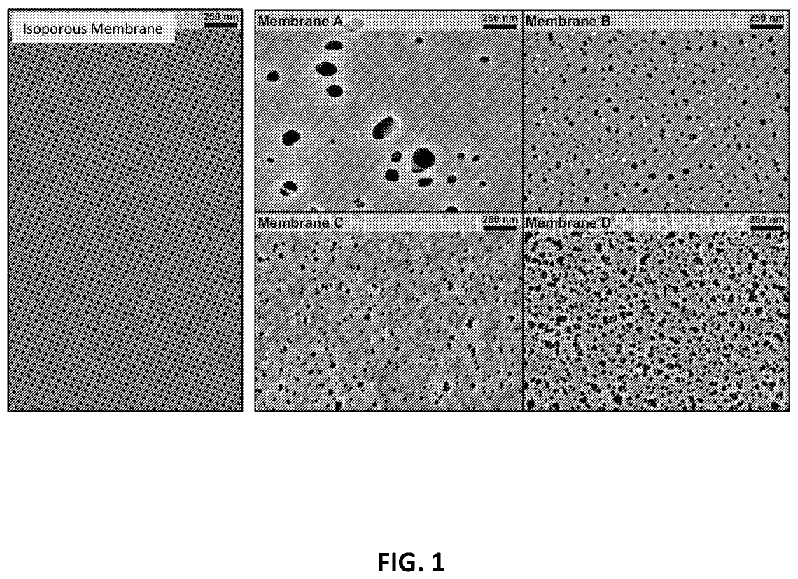

The isoporous membrane of embodiments of systems described herein is superior to other conventional, commercially available membranes due to the homogeneity of the pore size and distribution throughout the entirety of the membrane structure. FIG. 1 shows images comparing an isoporous membrane according to of embodiments of systems described herein to commercially available membranes (Membrane A, Membrane B, Membrane C, and Membrane D) claiming to have pore size and distribution equivalent to that of the isoporous membrane. As seen in FIG. 1, the pore size and distribution of the isoporous membrane is more uniform compared to Membranes A-D.

In an embodiment, the isoporous membrane comprises a uniform pore distribution, wherein pores have a uniform pore size. In an embodiment, the uniform pore size is in a range of 30 nm and comprises a pore size distribution where a maximum diameter divided by a minimum diameter is less than 3 nm. In an embodiment, the pore size of the isoporous membrane is selected based on a size of EV particles to be filtered from a biofluid. In an embodiment, the size of particles to be filtered from the biofluid is in a range of 50 nm to 250 nm. In an embodiment, the size of particles to be filtered from the biofluid is in a range of 80 nm to 150 nm. In an embodiment, the membrane does not require chemical or surface modifications.

In an embodiment, the membrane is formed of one or more polymer materials. In an embodiment, the one or more polymer materials comprise one or more of poly((4-vinyl)pyridine), poly((2-vinyl) pyridine), poly(ethylene oxide), poly(methacrylates) such as poly(methacrylate), poly(methyl methacrylate), and poly(dimethylethyl amino ethyl methacrylate), poly(acrylic acid), and poly(hydroxystyrene) poly((4-vinyl)pyridine, poly(styrene) and poly(alpha-methyl styrene), polyethylene, polypropylene, polyvinyl chloride, and polytetrafluoroethylene, poly(isoprene), poly(butadiene), poly(butylene), and poly(isobutylene), b-, poly(styrene)-b-poly((4-vinyl)pyridine), poly(styrene)-b-poly((2-vinyl) pyridine), poly(styrene)-b-poly(ethylene oxide), poly(styrene)-b-poly(methyl methacrylate), poly(styrene)-b-poly(acrylic acid), poly(styrene)-b-poly(dimethylethyl amino ethyl methacrylate), poly(styrene)-b-poly(hydroxystyrene), poly(α-methyl styrene)-b-poly((4-vinyl)pyridine), poly(α-methyl styrene)-b-poly((2-vinyl) pyridine), poly(α-methyl styrene)-b-poly(ethylene oxide), poly(α-methyl styrene)-b-poly(methyl methacrylate), poly(α-methyl styrene)-b-poly(acrylic acid), poly(α-methyl styrene)-b-poly(dimethylethyl amino ethyl methacrylate), poly(α-methyl styrene)-b-poly(hydroxystyrene), poly(isoprene)-b-poly((4-vinyl)pyridine), poly(isoprene)-b-poly((2-vinyl) pyridine), poly(isoprene)-b-poly(ethylene oxide), poly(isoprene)-b-poly(methyl methacrylate), poly(isoprene)-b-poly(acrylic acid), poly(isoprene)-b-poly(dimethylethyl amino ethyl methacrylate), poly(isoprene)-b-poly(hydroxystyrene), poly(butadiene)-b-poly((4-vinyl)pyridine), poly(butadiene)-b-poly((2-vinyl) pyridine), poly(butadiene)-b-poly(ethylene oxide), poly(butadiene)-b-poly(methyl methacrylate), poly(butadiene)-b-poly(acrylic acid), poly(butadiene)-b-poly(dimethylethyl amino ethyl methacrylate), and poly(butadiene)-b-poly(hydroxystyrene), poly(isoprene-b-styrene-b-4-vinylpyridine), poly(isoprene)-b-poly(styrene)-b-poly((4-vinyl)pyridine), poly(isoprene)-b-poly(styrene)-b-poly((2-vinyl) pyridine), poly(isoprene)-b-poly(styrene)-b-poly(ethylene oxide), poly(isoprene)-b-poly(styrene)-b-poly(methyl methacrylate), poly(isoprene)-b-poly(styrene)-b-poly(acrylic acid), poly(isoprene)-b-poly(styrene)-b-poly(dimethylethyl amino ethyl methacrylate), poly(isoprene)-b-poly(styrene)-b-poly(hydroxystyrene), poly(isoprene)-b-poly(α-methyl styrene)-b-poly((4-vinyl)pyridine), poly(isoprene)-b-poly(α-methyl styrene)-b-poly((2-vinyl) pyridine), poly(isoprene)-b-poly(α-methyl styrene)-b-poly(ethylene oxide), poly(isoprene)-b-poly(α-methyl styrene)-b-poly(methyl methacrylate), poly(isoprene)-b-poly(α-methyl styrene)-b-poly(acrylic acid), poly(isoprene)-b-poly(α-methyl styrene)-b-poly(dimethylethyl amino ethyl methacrylate), poly(butadiene)-b-poly(styrene)-b-poly((4-vinyl)pyridine), poly(butadiene)-b-poly(styrene)-b-poly((2-vinyl) pyridine), poly(butadiene)-b-poly(styrene)-b-poly(ethylene oxide), poly(butadiene)-b-poly(styrene)-b-poly(methyl methacrylate), poly(butadiene)-b-poly(styrene)-b-poly(acrylic acid), poly(butadiene)-b-poly(styrene)-b-poly(dimethylethyl amino ethyl methacrylate), poly(butadiene)-b-poly(styrene)-b-poly(hydroxystyrene), poly(butadiene)-b-poly(α-methyl styrene)-b-poly((4-vinyl)pyridine), poly(butadiene)-b-poly(α-methyl styrene)-b-poly((2-vinyl) pyridine), poly(butadiene)-b-poly(α-methyl styrene)-b-poly(ethylene oxide), poly(butadiene)-b-poly(α-methyl styrene)-b-poly(methyl methacrylate), poly(butadiene)-b-poly(α-methyl styrene)-b-poly(acrylic acid), poly(butadiene)-b-poly(α-methyl styrene)-b-poly(dimethylethyl amino ethyl methacrylate), and poly(butadiene)-b-poly(styrene)-b-poly(hydroxystyrene).

The system for isolating, separating, and concentrating EVs further comprises an ultrafiltration device. The isoporous membrane is compatible for use with a variety of ultrafiltration devices. In an embodiment, the system comprises an isoporous membrane configured to be used in the ultrafiltration device. The ultrafiltration device may be configured to perform diafiltration. In an embodiment, the ultrafiltration device comprises a fixed volume ultrafiltration device. A nonlimiting example of a fixed volume ultrafiltration device includes the AMICON Stirred Cell diafiltration device (available from Merck KGaA, Darmstadt, Germany). Such a device allows for isolating, separating, and concentrating EVs without requiring centrifugation. The ultrafiltration device may have any suitable size. As nonlimiting examples, the ultrafiltration device may comprise a 50 mL fixed volume ultrafiltration device or a 400 mL fixed volume ultrafiltration device.

In an embodiment, the ultrafiltration apparatus comprises a fixed volume ultrafiltration device. In embodiments, the fixed volume ultrafiltration device allows for scalability up to 400 mL. The fixed volume ultrafiltration device is easy-to-clean, can perform multiple modes of ultrafiltration including diafiltration, and uses air pressure as the driving separation force instead of fluid flow (which can alter EV integrity).

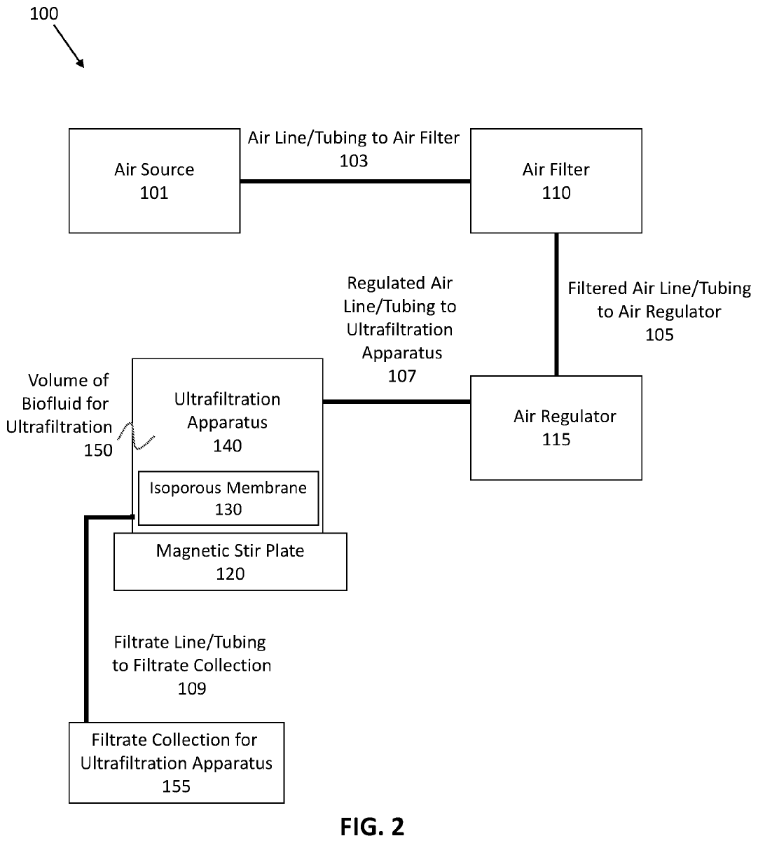

FIG. 2 shows an embodiment of a system for separating, isolating, and concentrating extracellular vesicles (EVs) 100 according to the present disclosure. As a nonlimiting example, the system may be configured to carry out ultrafiltration applications for downstream processing of EVs from spent cell culture media. The system 100 may comprise a simple configuration or setup. The system 100 may comprise an ultrafiltration device 140; an isoporous membrane 130 configured for use in the ultrafiltration device 140; and a collection container 155 for collecting filtrate from the ultrafiltration device 140.

The ultrafiltration device 140 may comprise a fixed volume ultrafiltration device. A biofluid may be introduced to the fixed volume ultrafiltration device for processing, wherein processing of the biofluid comprises separating, isolating, and concentrating EVs from the biofluid. The ultrafiltration device may be any suitable size or volume. As a nonlimiting example, the ultrafiltration device may comprise a 50 mL fixed volume ultrafiltration device.

The system 100 may comprise a simple ultrafiltration configuration setup that uses the fixed volume ultrafiltration device with flow driven by air pressure. An air source 101, such as an in house air line, may provide air to the fixed volume ultrafiltration device 140. The system may further comprise an air filter 110, such as a three stage air filter. The system 100 may further comprise an air regulator 115, such as a sensitive digital air regulator. Air from the air source 101 may travel through an air line or tubing 103 to the air filter 110. Filtered air from the air filter 110 may travel though a filtered air line or tubing 105 to the air regulator 115. Regulated air may travel from the air regulator 115 through a regulated air line or tubing 107 to the ultrafiltration device or apparatus 140.

The ultrafiltration device 120 may be disposed on a magnetic stir plate 120. Filtrate from solution 150 in the ultrafiltration device 140 may travel though a filtrate line or tubing 109 to a filtrate collection container 155. Air lines or tubing and filtrate line or tubing may comprise any tubing suitable for use with biofluids, such as sterilized plastic tubing.

In an embodiment, the ultrafiltration apparatus comprises a tangential flow filtration apparatus. The tangential flow filtration device allows for large working volumes, precision control of pressure and flow rate, and can be automated. In an embodiment, the tangential flow filtration device comprises a flat membrane flow cell.

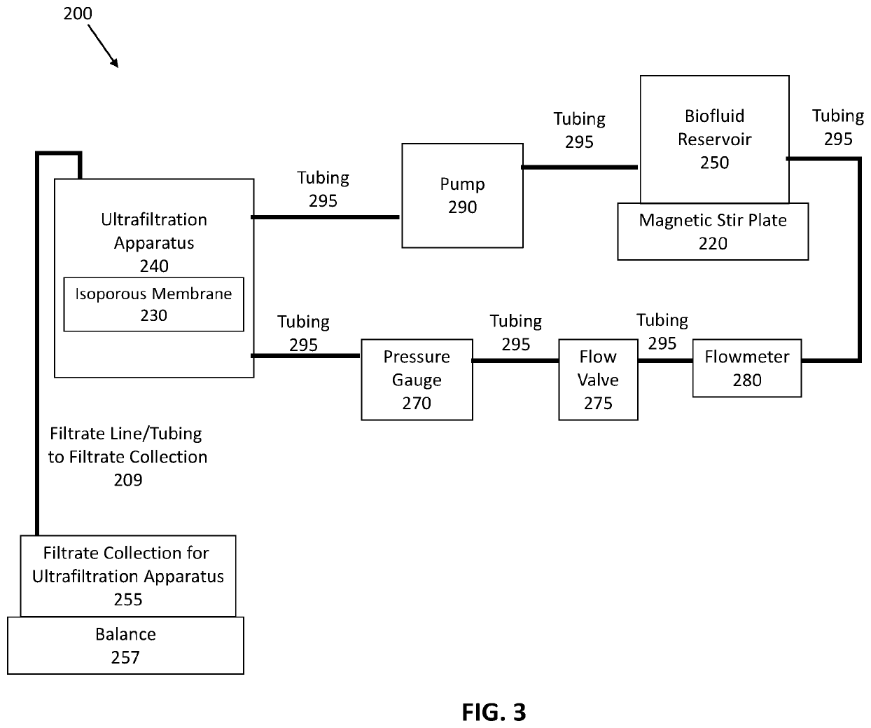

FIG. 3 shows an embodiment of a system for separating, isolating, and concentrating extracellular vesicles (EVs) 200 according to the present disclosure. For example, the system may be configured to carry out ultrafiltration applications for downstream processing of EVs from spent cell culture media. The system 200 may comprise a simple configuration or setup comprising an ultrafiltration device 240; an isoporous membrane 230 configured for use in the ultrafiltration device 240; and a collection container 255 for collecting filtrate from the ultrafiltration device 240.

The ultrafiltration device 240 may comprise a tangential flow filtration device (TFF), such as a flow cell. Biofluid from reservoir 250 may travel to the ultrafiltration device 240 comprising an isoporous membrane 230 coupled to the ultrafiltration device 240 through tubing 295. A flowmeter 280, flow valve 275, pressure gauge 270, or a combination thereof may be disposed or arranged on the tubing 295 between the reservoir 250 and the ultrafiltration device 240. The solution travels through the ultrafiltration device and is processed. Outputs from the ultrafiltration device 240 comprise filtrate (which travels through tubing 209 to the filtrate collection container 255) and biofluid permeate (which travels from the ultrafiltration device to pump 290 and back to the biofluid reservoir 250). As such, the ultrafiltration device processes the biofluid, wherein EVs in the form of the filtrate are separated, isolated, and concentrated from the biofluid. The ultrafiltration device 240 may be any suitable size or volume.

The reservoir of biofluid 250 for ultrafiltration may optionally be disposed on a magnetic stir plate 220. The isolated EVs or filtrate processed by the ultrafiltration device 240 from the biofluid reservoir 250 may travel though a filtrate line or tubing 209 to a filtrate collection container 255. The filtration collection container 255 may optionally be disposed on a balance or scale 257 to weigh or monitor the amount of filtrate collection. Tubing 295 designates tubing where biofluid and processed and/or recirculated biofluid flows through. Tubing 295 and filtrate line or tubing 209 may comprise any tubing suitable for use with biofluids, such as sterilized plastic tubing.

### EXAMPLES

### Example 1

Example 1 investigated the performance of isoporous membranes and compared the performance of the isoporous membranes to conventional membranes of equivalent geometry and approximate pore size. The specific flux of water was determined through three isoporous membranes and three respective equivalent conventional membranes. Example 1 further included a comparison of the ability of the membranes to concentrate spent media containing mock extracellular vesicles (EVs) using an ultrafiltration device.

Materials for Example 1 included isoporous membranes (having 5, 10, and 30 nm pore sizes, \acute{ø} 44.5 mm), conventional membranes (Millipore Sigma Biomax Membranes having 30, 50, and 100 kDa pore sizes, \acute{ø} 44.5 mm), mock extracellular vesicle containing media wherein the EV mimic included 100 nm polystyrene latex standard beads from Malvern, fixed volume ultrafiltration device, magnetic stir plate, digital air regulator (0.00 PSI precision), a three stage air filter, NanoSight N3000 (Malvern Panalytical), and QuantiPro™ BCA Assay Kit (Sigma Aldrich).

### Experimental Setup

An in house air-line was outfitted to the fixed volume ultrafiltration device with a three stage air filter leading to a sensitive digital air regulator. The regulator had been prior calibrated and adjusted to reach 2.00 PSI. The filtrate outlet line of the stirred cell was fixed into a graduated cylinder as a collection container for measurable collection of water filtrate. The stirred cell was placed on top of the magnetic stir plate and secured with a ring stand clamp. All experiments used an analog setting of 3 on the stir plate, which is approximately 100 rpm.

### Specific Flux Measurement

Each membrane was assembled into the stirred cell and pre-conditioned with 10 mL of MilliQ water. Once fully conditioned, the set-up was fully assembled and air pressure was engaged at 2.00±0.005 PSI. Time was recorded for every 5 mL of water filtrate collected until 50 mL was achieved or one hour of time expired.

### Mock EV Concentration

Each membrane was installed into the stirred cell as instructed by the user manual and washed with PBS to remove preservatives. Mock EV media was then pipetted into the fixed volume ultrafiltration device and concentrated by half (experiment concluded when a set volume of permeate was collected in a graduated cylinder). Permeate and concentrate were collected and stored at 4° C. until needed for subsequent testing. Each membrane type was tested with two independent trials (n=2 per group). EV concentration was measured using Nanoparticle Tracking Analysis on the NanoSight N3000 (n=6; three measurements of two independent samples) following our prior described protocol for EV measurements. Protein concentration was measured using colorimetric BCA assay kit following the manufacturer's protocol.

### Specific Flux Measurement and Calculation

Preliminary evaluations demonstrate the specific flux values provided by isoporous membranes are similar to the measured results for water (Table 1 and Table 2). No values are available in the technical documents for the conventional membranes. After some preliminary calculations, it was noted that specific flux was not constant over time. After eliminating the first time point, a linear regression was performed to understand if the loss in flux over time was constant. In general, the flux is not linearly dropping over time as indicated by low correlation coefficients. This was also confirmed by assessing the change in slope (second derivative calculations, not shown), which shows a decrease in flux change over time (i.e. the flux is decreasing over the experiment, but the rate of decrease is less over time). This could be attributed to two factors, the first being membrane fouling and pore obstruction, and the second being the assistance of gravity with an increased fluid mass upstream from the membrane.

For an ultrafiltration device, such as a diafiltration device, having a fixed volume that relies on air pressure to drive fluid flow, it is not unexpected that a better flux is achieved when more fluid is occupying space in the chamber rather than air. Although this phenomenon is not significantly affecting this small 50 mL system overall, it should be noted that if a large apparatus designed like a stirred cell should be considered for future experiments, the relationship between mass transfer and volume of fluid in the chamber may have a significant impact on flux.

TABLE 1
Details of the membranes tested

| | | Membrane | | |
|---|---|---|---|---|
| | Pore Size | Surface | Filtrate | Filtration |
| Membrane | (nm) | Area (m²) | Vol (L) | Time (hr) |
| Conventional 100 kDa | 100 | 0.00445 | 0.05 | 0.35972 |
| Conventional 50 kDa | 5 | 0.00445 | 0.05 | 0.39583 |
| Conventional 30 kDa | <5 | 0.00445 | 0.05 | 0.53750 |
| Isoporous 30 nm | 30 | 0.00445 | 0.05 | 0.15222 |
| Isoporous 10 nm | 10 | 0.00445 | 0.05 | 0.55972 |
| Isoporous 5 nm | 5 | 0.00445 | 0.05 | >1 |

TABLE 2
Specific flux of water for membranes
(Data shown mean + standard deviation)

| | Expected Specific Flux | Measured Specific |
|---|---|---|
| Membrane | (LMH/bar) | Flux (LMH/bar) |
| Conventional 100 kDa | — | 493 ± 9 |
| Conventional 50 kDa | — | 425 ± 10 |
| Conventional 30 kDa | — | 310 ± 3 |
| Isoporous 30 nm | 1218 | 1106 ± 23 |
| Isoporous 10 nm | 395 | 304 ± 9 |
| Isoporous 5 nm | 148 | 103 ± 5 |

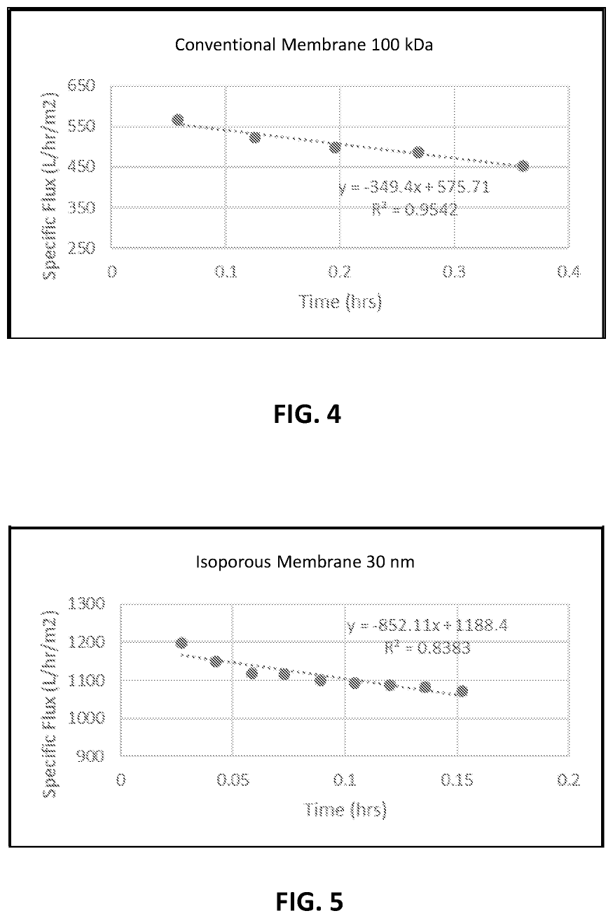

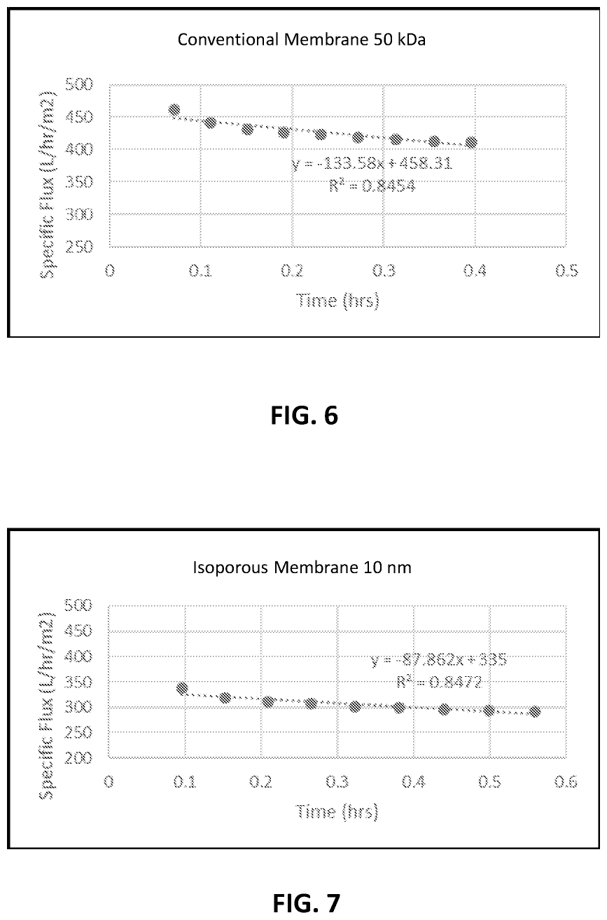

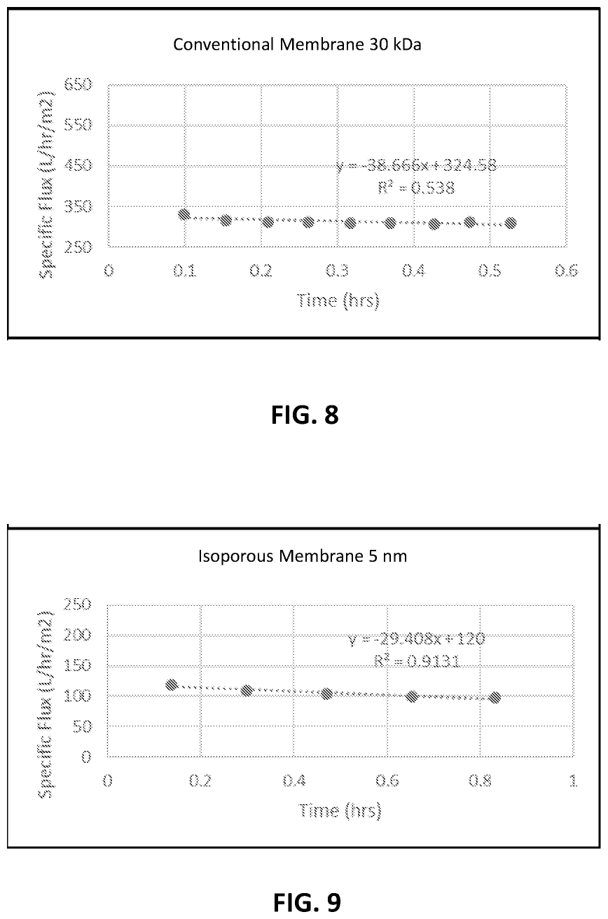

The linear regression analysis of specific flux of water over time through the respective conventional and isoporous membranes are shown in FIGS. 4-9. FIG. 4 shows linear regression analysis of specific flux of water for a conventional 100 kDa membrane, FIG. 5 shows linear regression analysis of specific flux of water for an isoporous 30 nm membrane, FIG. 6 shows linear regression analysis of specific flux of water for a conventional 50 kDa membrane, FIG. 7 shows linear regression analysis of specific flux of water for an isoporous 10 nm membrane, FIG. 8 shows linear regression analysis of specific flux of water for a conventional 30 kDa membrane, and FIG. 9 shows linear regression analysis of specific flux of water for an isoporous 5 nm membrane.

### Mock EV Concentration and Protein Elimination

Not unexpectedly, filtration of mock EV media was significantly more time consuming than the water flux experiment. The membranes having the smallest pore sizes showed flow rates of less than 0.3 mL/min. Both membrane types showed significant fouling on the surface when removed from the ultrafiltration device. Surfaces of the conventional membranes were fouled with a brown color while the isoporous membranes showed red-colored fouling. The fouling observation may be due to the isoporous membrane pore size being more precise than the conventional membrane pore size where capture of phenol red and other components is plausible. Different surface chemistries on each of the membranes may also explain selective capture of components on the surfaces.

To achieve optimal performance, the membranes must allow excess fluid, proteins, and other contaminates to permeate through the membrane while retaining the EVs without compromising vesicle quality.

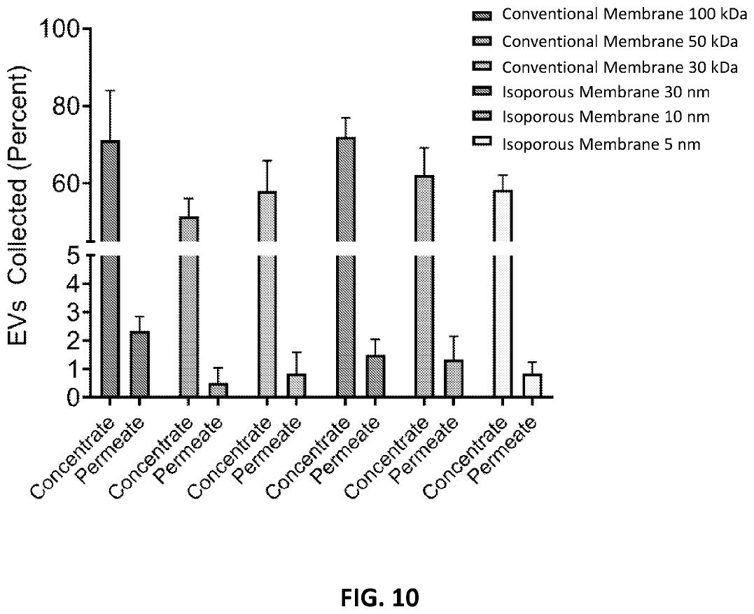

FIG. 10 shows the quantitation of collected EVs in the concentrate and permeate for each membrane tested. Data shown mean±standard deviation. As shown in FIG. 10, all isoporous membranes and conventional membranes had excellent performance in regard to preventing EV permeation through the membrane. All membranes demonstrated less than 2% EVs in the permeate solutions from the starting media. All membranes demonstrated a retention of >50% of EVs s from the solution. The isoporous membranes demonstrated a strong negative correlation between pore size and EV retention with significant loss of EVs with the smaller pore sizes. Conventional membranes were similar, except for the mid pore size which may be due to limited sample size. Regardless, the percent loss of EVs was significant, but less detrimental than other collection/purification methods used in previous experiments.

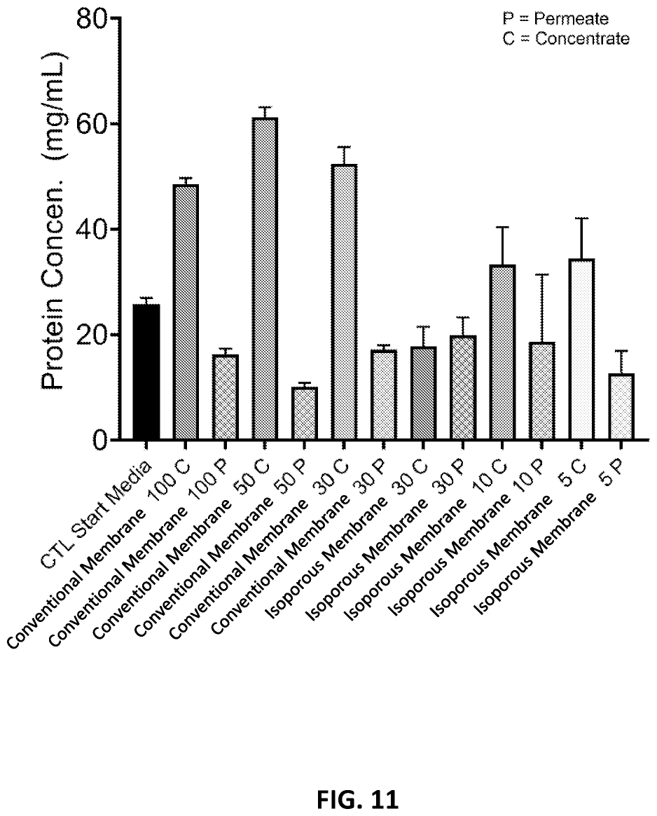

FIG. 11 shows the quantitation of protein via BCA assay. Data shown mean standard deviation. As shown in FIG. 11, performance in regard to purification was poor for conventional membranes. The protein concentration for all membrane pore sizes more or less doubled in the concentrate samples with low readings in the permeate samples. The isoporous membranes were more successful in purification, allowing more protein to flow through the membrane into the permeate solution. This was especially evident with the isoporous 30 nm pore size membrane where the protein concentration of the permeate is higher than the concentrate solution.

The experimental specific flux measured and calculated for each isoporous membrane was found to be satisfactorily similar to the data sheets provided by the seller. EV retention in the collection chamber was overall satisfactory compared to other methods of collection/purification, such as spin columns and polymer precipitation, among others. Isoporous membranes outperformed conventional commercial membranes for both EV retention and protein elimination. The isoporous membrane having a 30 nm pore size was the top performer.

### Example 2

Example 2 was performed to investigate the ability of isoporous membranes to separate and concentration true EVs from spent culture media. Example 2 expanded upon the Mock EV study performed as a proof of concept. A preliminary assessment of scaling up by a factor of 8 was investigated, and the scale-up was from a 50 mL ultrafiltration (diafiltration) device to a 400 mL ultrafiltration (diafiltration) device.

Materials for Example 2 included isoporous membranes having 30 nm pore sizes, \acute{ø} 44.5 mm and 76 mm, Vero Cells (ATCC), complete media for serum free Vero culture, TC-treated CellCube-10 (8500 cm²) with standard vent caps, TC-treated CellSTACK-5 (3180 cm²) with standard vent caps, Ascent Alpha Bioreactor (6780 cm²), 50 mL and 400 mL fixed volume ultrafiltration devices, magnetic stir plate, digital air regulator (0.00 PSI precision), a three stage air filter, graduated cylinders, NanoSight N3000 (Malvern Panalytical), and QuantiPro™ BCA Assay Kit (Sigma Aldrich).

### Cell Culture

Vero cells were cultured in the CellSTACK-5 (2D static culture), CellCube-10 (2D perfusion culture), and the Ascent prototype (2D perfusion culture) in conditions as described in Table 3. After the indicated expansion time, spent media was collected and frozen at −80° C. until testing.

TABLE 3
Cell culture vessels and conditions investigated
for Vero cells EV production

| | CellSTACK - 5 | | CellCube - 10 | | Ascent | |
|---|---|---|---|---|---|---|
| Cell culture vessel | (2D Static) | | (2D Perfusion) | | (2D Perfusion) | |
| Growth Area | 3180 | cm² | 8500 | cm² | 6780 | cm² |
| Media Volume | 1.5 | L | 3.2 | L | 690 | mL |
| Seeding Density | 30 | K/cm² | 17 | K/cm² | 15 | K/cm² |
| Perfusion Rate | N/A | | 0.2 | L/min | PO₂ Driven | |
| Expansion Time | 4 | Days | 6/6 | Days | 3/6 | Days |

### EV Separation and Concentration

Membranes were pre-conditioned with PBS. Spent EV media was pipetted into the fixed volume ultrafiltration device, concentrated by half, and the permeate was collected. Then a buffer was flowed into the fixed volume ultrafiltration device and the diluted concentrate was concentrated by half again and permeate was collected. This was repeated for a total of 5 cycles. The final concentrate was collected from the fixed volume ultrafiltration device and analyzed via NTA (n=2 per membrane, 3 independent replicates). Protein concentration was measured via BCA for all collected permeates and the final concentrate (n=2 per membrane, 3 independent replicates). For scaling up to the 400 mL fixed volume ultrafiltration device, the same protocol was used with volume adjustments of spent EV media and concentration was performed for 3 cycles.

### Comparison of True EV Separation and Concentration to Mock EV Results

Example 1 used latex polystyrene calibration standard beads with a tight size distribution of 100 nm to simulate EVs in a protein enriched media. Due to the difference in physiochemical properties of the calibration beads compared to true EVs, it had to be confirmed that the isoporous membranes would separate and concentrate true EVs with similar success to that of the mock EVs.

As the results of Example 2 show, the isoporous membrane can support true EV purification from spent cell culture media using the stirred cells. A greater than 90% reduction in proteins content and a retention of greater than 80% of EVs from starting culture was observed. With these results, no surface chemistry modifications or other experimental modifications are likely to be necessary when scaling up or when shifting to a Tangential Flow Filtration (TFF) system.

### Vero EV Characteristics and Protein Reduction

Cell culture vessels larger than T-75 and T-25 vessels were explored in Example 2. A cell type other than human mesenchymal stem cells was explored, as Vero (kidney epithelial cells), which are often used for the study of EV assisted viral transduction, were explored in Example 2. The objective of Example 2 included ensuring separation and concentration of EVs that are less than 200 nm in diameter, as literature suggests that small EVs are the most likely subcategory of EVs that will obtain the relevant therapeutic molecules and messages for clinical applications.

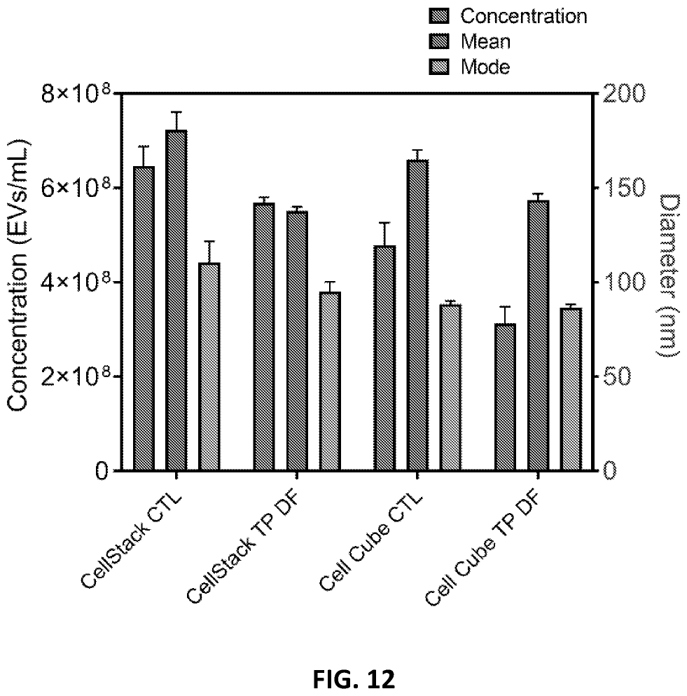

FIG. 12 shows the NTA analysis of Vero EV concentration and size from CellSTACK (4 day expansion) and CellCube (6 day expansion) vessels. Data shown mean standard deviation, n=2 independent samples, 3 triplicate readings per group.

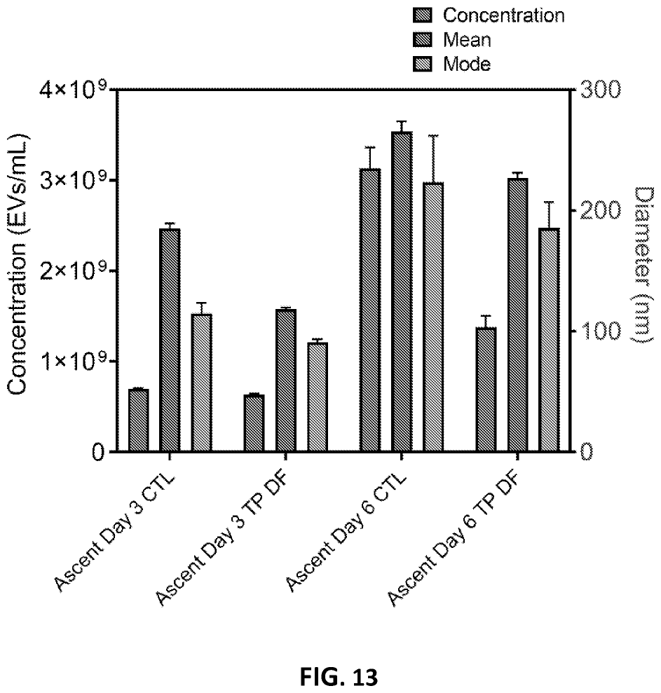

FIG. 13 shows the NTA analysis of Vero EV concentration and size from the Ascent prototype at Day 3 and 6. Data shown mean±standard deviation, n=2 independent samples, 3 triplicate readings per group.

As shown in FIG. 12 and FIG. 13, the vessels supported the production of about 10⁸-10⁹ EVs/mL. The majority of EVs collected had a mode diameter of about 80-120 nm, with the exception of the EVs collected from the Day 6 Ascent vessel. It is highly likely that the Ascent Day 6, which relies on re-circulation perfusion, resulted in decreased quality and quantity of EVs. Therefore, EV production should only be performed in perfusion vessels for up to 3 Days, as re-circulation does not provide appropriate conditions to maximize quality EV production.

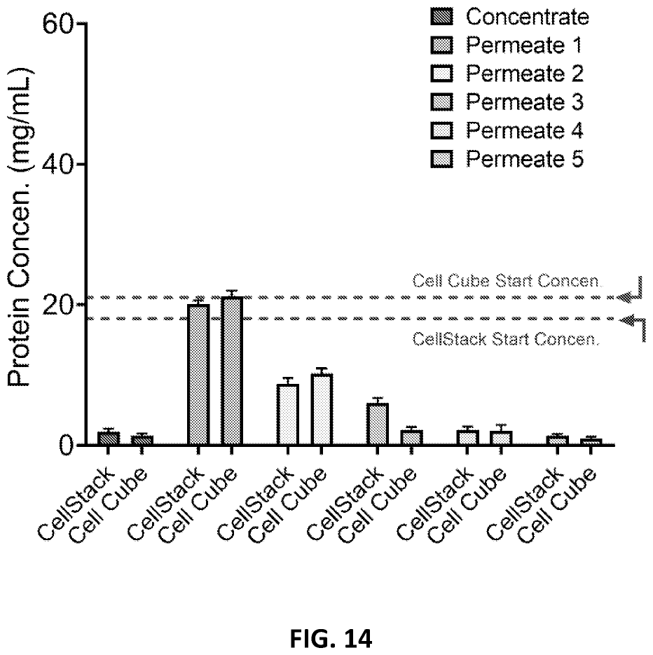

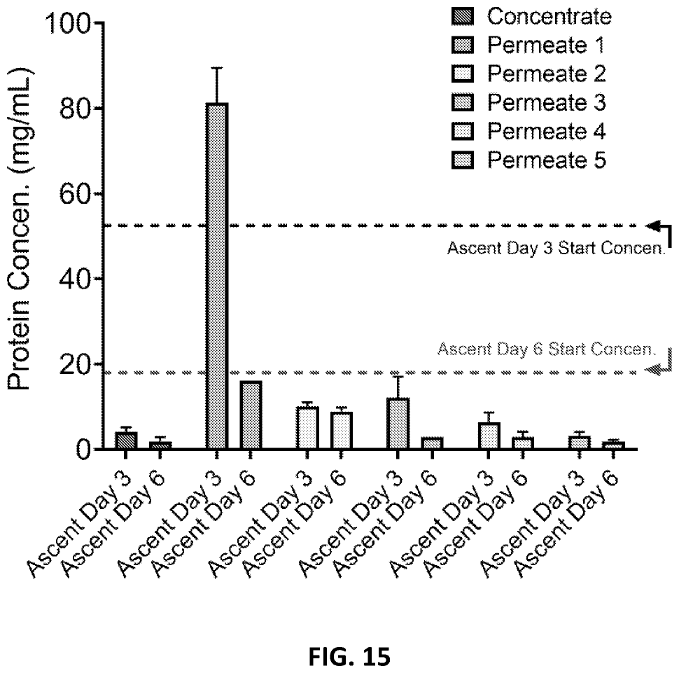

FIG. 14 and FIG. 15 demonstrate the protein reduction achieved using the diafiltration device. FIG. 14 shows the protein analysis via BCA assay of final EV concentrate and subsequent permeates from a static cell culture vessel and a perfusion cell culture vessel through diafiltration. The static cell culture vessel used included CellSTACK culture vessels (Corning Incorporated, Corning, NY) and the perfusion cell culture vessel used included CellCube culture vessels (Corning Incorporated, Corning, NY). Data shown mean±standard deviation, n=3 independent samples per group. FIG. 15 shows the protein analysis via BCA assay of final EV concentrate and subsequent permeates from a perfusion vessel such as the Ascent prototype fixed bed bioreactor available from (Corning Incorporated, Corning, NY) (Day 3 and Day 6 expansion) through the diafiltration. Data shown mean±standard deviation, n=3 independent samples per group.

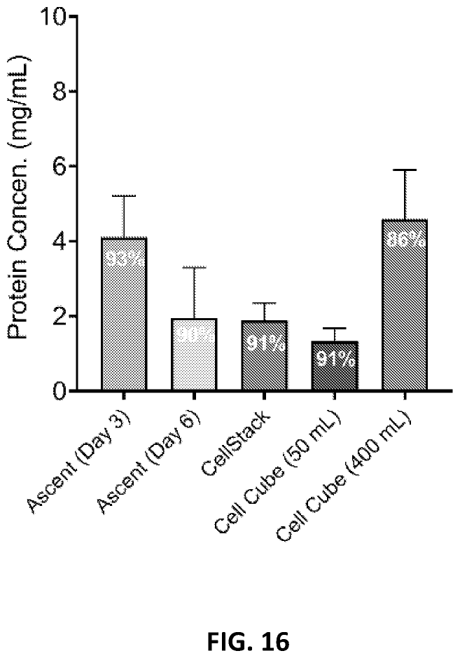

FIG. 16 shows a comparison of protein reduction in final concentrate of Vero produced EVs. Data shown mean±standard deviation, n=6 independent samples per group. As summarized in FIG. 16, protein reduction averaged around 90% from the starting control spent media for each vessel type after diafiltration. As noted, the majority of protein is removed in the first three cycles, thus moving forward we will use three cycles instead of five cycles of diafiltration for larger volume experiments.

Scale-Up from 50 mL to 400 mL

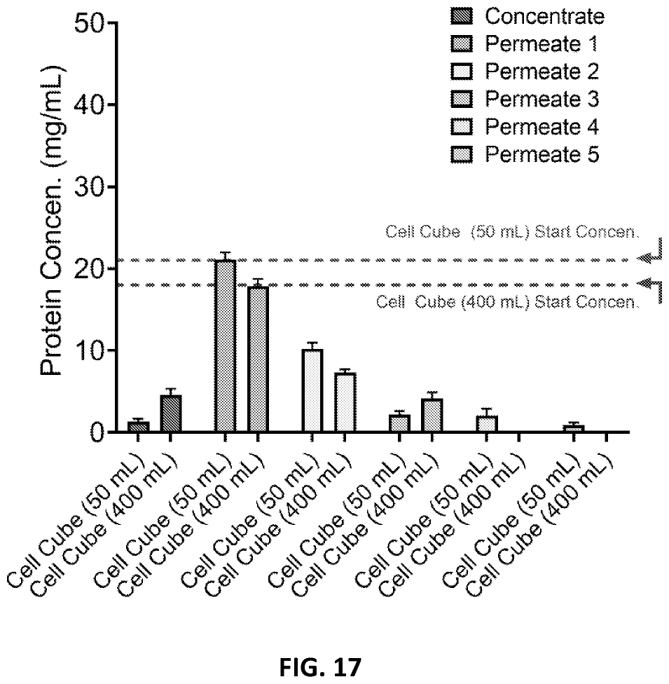

When scaling up from a 50 mL fixed volume ultrafiltration device to a 400 mL fixed volume ultrafiltration device, the isoporous membrane for the device increased in diameter from 44.5 mm to 76 mm. This is an increase in area of 70%, despite the increase in working volume being 8 times. It is not unexpected that differences should be observed when scaling up in a non-linear system. Similar protein reduction (91% and 86%) was achieved using the same media produced from the CellCube Day 6 experiment, as shown in FIG. 17. FIG. 17 shows the protein analysis via BCA assay of final EV concentrate and subsequent permeates from CellCube through the diafiltration. Data shown mean±standard deviation, n=3 independent samples per group.

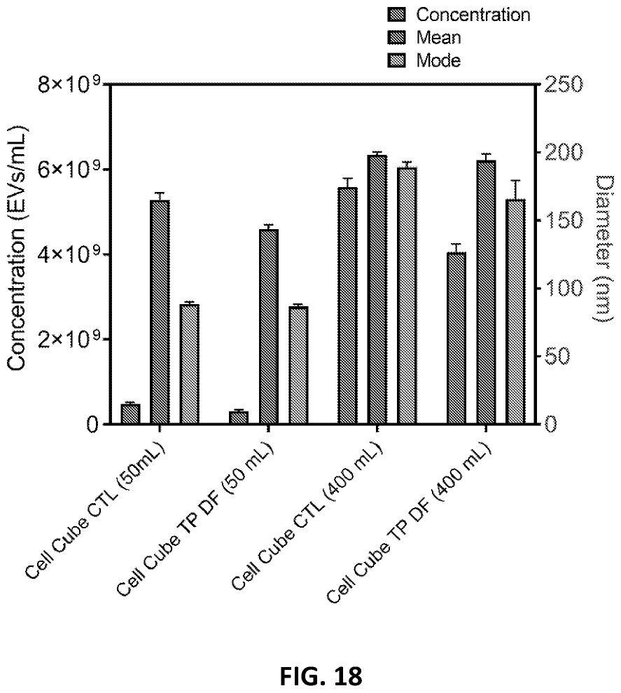

FIG. 18 shows NTA analysis of Vero EV concentration and size from CellCube vessels using the 50 mL and 400 mL fixed volume ultrafiltration devices. Data shown mean standard deviation, n=2 independent samples, 3 triplicate readings per group. FIG. 18 shows that larger diameter EVs (150-200 nm) were collected in the larger device (400 mL) compared to the smaller diameter EVs (80-90 nm) collected in the 50 mL device. One possibility for the observation is that the larger volume was able to more accurately represent the true population which is missed in the smaller volume. Another possibility for the observation is that the forces (air pressure) driving the filtration need to be modified in conjunction with the scale-up. Moreover, the 50 ml device had 5 cycles and lasted 3.5 hrs, while the 400 mL device had 3 cycles that lasted 6 hrs. Thus, the duration time for separating and concentrating was significantly longer for the larger volume, which could influence EV stability due to constant mixing.

Results of Example 2 indicated that the isoporous membrane can separate and concentrate true EVs with the same rigor as demonstrated with the mock EV proof of concept experiments. Scaling up to larger cell culture vessels may allow for clinically relevant production volumes for EV therapies.

### Example 3

Example 3 was performed to test the ability of isoporous membranes to separate and concentration true EVs from spent culture media. Prior work analyzed the concentration and purification of EVs using ultrafiltration, or diafiltration in the fixed volume ultrafiltration device with the largest working volume of 400 mL. Example 3 details methods and analyses of transitioning to using Tangential Flow Filtration (TFF) using a flat membrane flow cell kit.

Materials for Example 3 included isoporous membranes having 30 nm pore size, Vero Cells (ATCC), complete media for serum free Vero culture, TC-treated CellCube-10 (8500 cm²) with standard vent caps (Corning Incorporated, Corning, NY), TC-treated CellSTACK-5 (3180 cm²) with standard vent caps (Corning Incorporated, Corning, NY), Ascent Alpha Bioreactor (6780 cm²) (Corning Incorporated, Corning, NY), fixed volume ultrafiltration device in 50 mL and 400 mL sizes, magnetic stir plate, digital air regulator (0.00 PSI precision), a three stage air filter, graduated cylinders, NanoSight N3000 (Malvern Panalytical), QuantiPro™ BCA Assay Kit (Sigma Aldrich), and tangential flow filtration device.

### Tangential Flow Filtration (TFF)

A tangential flow filtration device may allow for operation of cross flow cells with small working and hold-up volumes. With a small total hold-up volume (up to 1 liter) in the test cell and circulation line, such a bench-scale cross or tangential flow filtration (TFF) system is ideal for filtering valuable feed solutions and/or small sample volumes. Separately, a bench-scale cross/tangential flow cell may provide fast and accurate performance data with minimal amounts of membrane, product, expense, or time wherein the design uses a flat sheet membrane to drive flow tangentially across the membrane. Other designs may use hollow fibers or spiral or folded membrane configurations to increase surface area.

### EV Separation, Concentration, and Characterization

Isoporous membranes were pre-conditioned with 500 mL of PBS running through the same pressure as the preceding experiment. Each membrane was independently run twice, once at 2 PSI and once at 30 PSI with 1 L of spent culture media from prior experiments (cell culture details can be found in Report 2 and Report 3). The flow was dictated by the pressure using the needle flow valve and the analog setting on the magnetic pump. Each run was complete when either 6 hours had passed, or the fluid volume was too low for the dip tube to draw the spent media into the pump inlet. Immediately after each run, volume measurements were taken for the concentrated EV solution, permeate, and fluid loss was calculated. To characterize EVs, Nanoparticle Tracking Analysis (NTA) was used to determine concentration and size distribution for the concentrate and determine if any particles passed through the membrane in the permeate. Colorimetric BCA protein assay was also used to determine protein concentration in permeate and concentrate.

### Comparison of Ultrafiltration, Diafiltration, and Tangential Flow Filtration

Three methods of concentrating and purifying extracellular vesicles were investigated: ultrafiltration, diafiltration, and tangential flow filtration. Though each method has advantages and challenges, it is clear that the isoporous membrane is more versatile and easier to work with compared to conventional, commercial membranes.

Both ultrafiltration and diafiltration use systems that drive flow directly from one side of the membrane to the other side of the membrane. The TFF system drives flow tangentially to the membrane using other fluid dynamics to drive undesired molecules out of solution. Unfortunately, significant challenges for the TFF system may have impacted the results, including incorrectly sourced parts, poor technical assistance, poor performing kit components, and availability of only two isoporous membranes of the same pore size. With these limitations in mind, the TFF system was not as successful at concentrating and purifying EVs as the diafiltration mode ultrafiltration.

The conventional, commercial membrane was unable to stably run in the TFF system at a low pressure such as 2 PSI. However, prior experiments demonstrated that lower pressures were found to result in high purity of the target population of EVs. The isoporous membrane was able to run in the TFF system stably at the equivalent 2 PSI pressure used in ultrafiltration and diafiltration. The TFF system also requires a run time to complete the full TFF cycle. The conventional commercial membrane at 30 PSI, and the isoporous membrane cycle times were greater than 6 hrs and were stopped before reaching maximum concentration. The isoporous membrane run at 30 PSI was completed in 5 hrs.

### Vero EV Characteristics and Protein Reduction

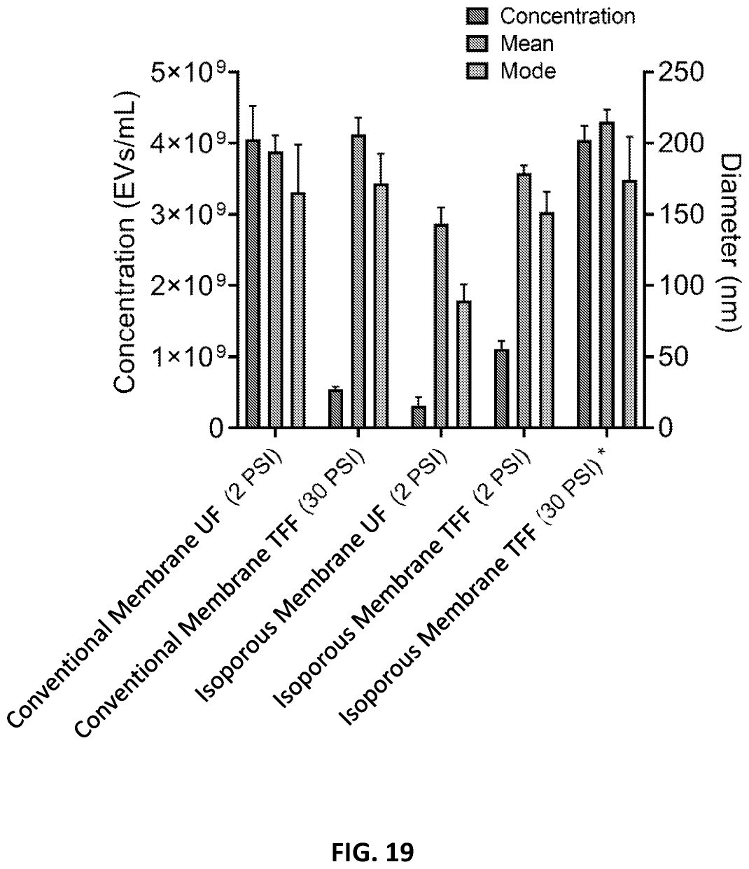

To understand the TFF performance at the two experimental pressures, data was extracted from the first pass of the diafiltration cycles that would be an appropriate mimic of a TFF cycle. FIG. 19 shows analysis of concentrated and purified EVs from NTA. Data shown mean±standard deviation, n≥6 per group. As shown in FIG. 19, the conventional competitive offering membrane showed increased concentration with ultrafiltration compared to TFF. The opposite result was noted with the isoporous membrane. That being said, the size distribution captured from the isoporous membrane using TFF was larger than the size distribution captured with ultrafiltration. As mentioned in prior reports, the goal is to capture EVs in the 80-150 nm range, which is where the best therapeutic potential lies. The data with the diafiltration shows a superior capture of this target range compared to simple ultrafiltration or TFF. Thus, though TFF performance in Example 3 was inferior to prior work in Example 1 and Example 2, the TFF performance still outperformed most conventional technologies.

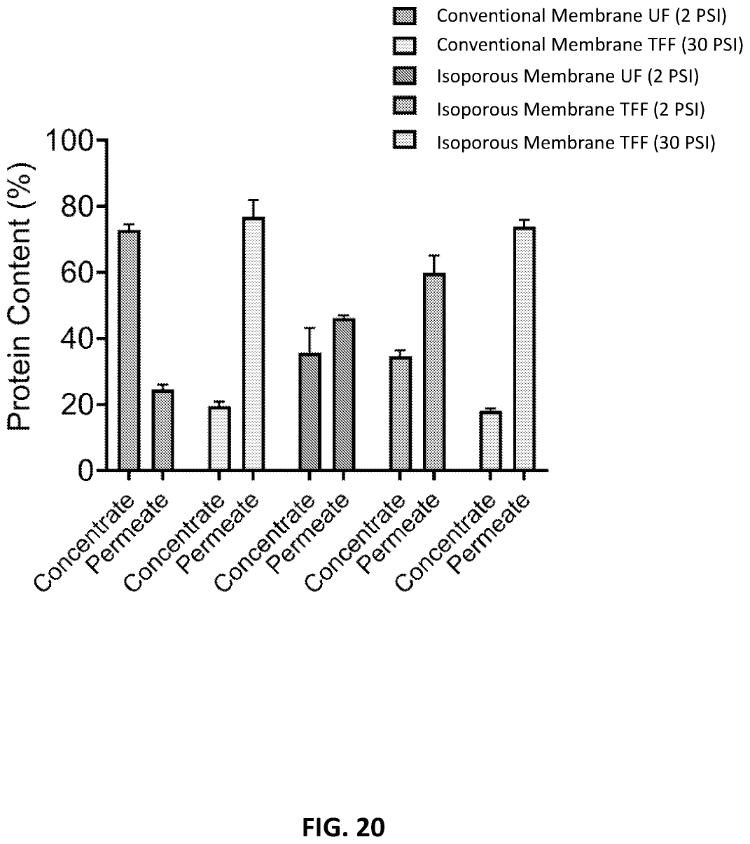

As shown in FIG. 20, better protein reduction was observed in TFF compared to simple ultrafiltration. FIG. 20 shows protein concentration analysis via BCA assay. Data shown mean±standard deviation, n=3 per group. This was not unexpected, as the fluid is cycled tangentially to the membrane exponentially more times than during ultrafiltration. Protein reduction observed for TFF was inferior to the diafiltration samples by 10-15%.

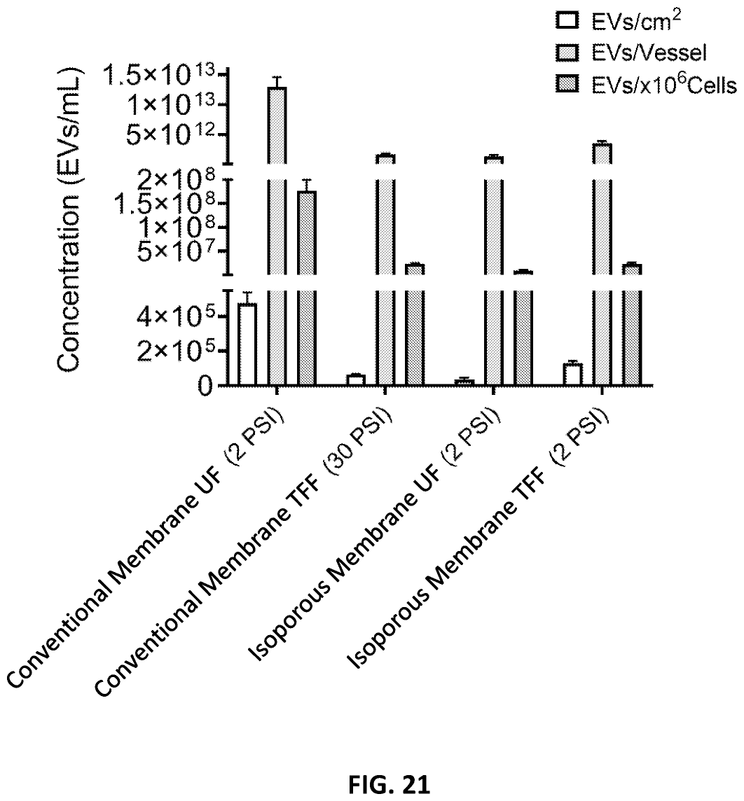

The conventional membranes showed a significant difference in EVs captured using ultrafiltration (diafiltration) compared to tangential flow filtration, as shown in FIG. 21. However, because the conventional membranes were sourced from different vendors due to the larger geometry required for the TFF unit, an equivalent performance cannot be guaranteed, despite the same pore size and material composition.

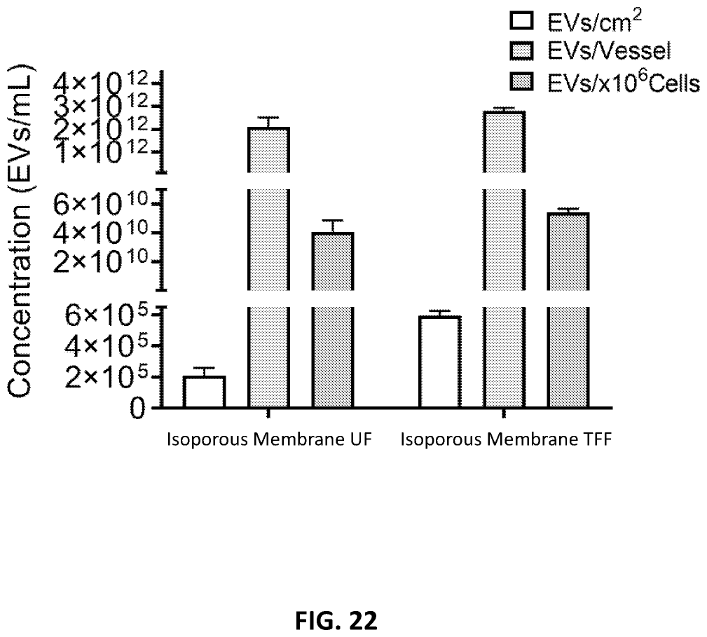

The total EVs captured using the isoporous membranes were not statistically different between UF and TFF, as indicated by FIG. 21 and FIG. 22. FIG. 21 shows a graphical image of total EV production from a perfusion culture device such as CellCube (Corning Incorporated, Corning, NY). Data shown mean±standard deviation, n=3 per group. FIG. 22 shows a graphical image of total EV production from a perfusion culture device such as Ascent fixed bed bioreactor (Corning Incorporated, Corning, NY). Data shown mean standard deviation, n=3 per group.

The limited sample size of isoporous membranes and the technical challenges of the TFF device may hinder complete understanding of membrane performance in the experiment. Though the isoporous membrane outperformed the conventional membranes, the isoporous membrane did not show improvement upon diafiltration results.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been illustrated in the accompanying Drawings and described in the foregoing Detailed Description, it should be understood that the disclosure is not limited to the disclosed embodiments, but is capable of numerous rearrangements, modifications and substitutions without departing from the disclosure as set forth and defined by the following claims.

1. A system for separating, isolating, and concentrating extracellular vesicles (EVs) comprising:
an ultrafiltration device;
an isoporous membrane configured for use in the ultrafiltration device; and
a collection container for collecting filtrate from the ultrafiltration device.

2. The system of claim 1, wherein filtrate comprises a concentration of EVs separated and isolated from a biofluid processed by the system.

3. The system of claim 2, wherein the membrane comprises a uniform pore distribution, wherein pores have a uniform pore size.

4. The system of claim 3, wherein the uniform pore size is in a range of 30 nm and comprises a pore size distribution where a maximum diameter divided by a minimum diameter is less than 3 nm.

5. The system of claim 2, wherein the pore size of the isoporous membrane is selected based on a size of EV particles to be filtered from a biofluid.

6. The system of claim 2, wherein the size of particles to be filtered from the biofluid is in a range of 50 nm to 250 nm.

7. The system of claim 2, wherein the size of particles to be filtered from the biofluid is in a range of 80 nm to 150 nm.

8. The system of claim 1, wherein the membrane does not require chemical or surface modifications.

9. The system of claim 1, wherein the membrane is formed of a material comprising one or more polymer materials.

10. The system of claim 1, wherein the ultrafiltration device comprises an automated ultrafiltration device.

11. The system of claim 10, wherein the ultrafiltration device system is configured to control pressure in the system.

12. The system of claim 10, wherein the ultrafiltration device is configured to control flow rate of fluid through the isoporous membrane by means of air pressure.

13. The system of claim 1, wherein the ultrafiltration device is configured to perform diafiltration.

14. The system of claim 1, wherein the ultrafiltration device comprises a fixed volume ultrafiltration device.

15. The system of claim 14, wherein the fixed volume ultrafiltration device comprises a volume in a range of 50 mL to 200 L.

16. The system of claim 14, wherein air pressure is the separation force.

17. The system of claim 16, further comprising an air source, air filter, air regulator, or combination thereof.

18. The system of claim 1, wherein the ultrafiltration device comprises a tangential flow filtration device.

19. The system of claim 1, further comprising a reservoir comprising a biofluid to be processed.

20. The system of claim 1, further comprising a pump, flow valve, flowmeter, pressure gauge, or combination thereof.

21. The system of claim 1, wherein the system is scalable.
